(19) United States
(12) Patent Application Publication
(10) Pub. No.: US 2017/0275572 A1
(43) Pub. Date: Sep. 28, 2017

(54) COMPOSITIONS FOR PHOTODYNAMIC CONTROL OF INFECTION

(71) Applicant: PUREPURGE, INC., VALENCIA, CA (US)

(72) Inventor: GEORGE V. GARNER, GREATER MANCHESTER (GB)

(73) Assignee: PUREPURGE, INC., VALENCIA, CA (US)

(21) Appl. No.: 15/506,918

(22) PCT Filed: Mar. 19, 2015
(86) PCT No.: PCT/US2015/021440

§ 371 Date: Feb. 27, 2017

Related U.S. Application Data
(60) Provisional application No. 62/047,803, filed on Sep. 9, 2014.

(51) Int. Cl. C11D 11/00; C11D 3/40; C11D 7/26; A61K 33/30; C11D 3/39; A61K 31/409; A61K 33/06; C11D 3/48; C11D 7/50

(52) U.S. Cl. CPC C11D 11/007; C11D 3/48; C11D 3/40; C11D 7/265; C11D 7/5009; C11D 3/3947; A61K 31/409; A61K 33/06; A61K 33/30

(57) ABSTRACT

The present invention relates to compositions for the photodynamic control of micro-organisms wherein the compositions comprise a photosensitiser which comprises a dyestuff and produces singlet oxygen when irradiated by means of light for the disinfection and sterilization of materials, commodities and surfaces contaminated with one or more species of micro-organisms including bacteria, fungi, algae, yeasts, bacterial spores and fungal spores.

### CROSS REFERENCE TO RELATED APPLICATION

The present application and invention claims priority to U.S. Provisional Application No. 62/047,803 filed on Sep. 9, 2014, the contents of which are incorporated by reference herein for all purposes.

### BACKGROUND OF THE INVENTION

Technical Field

The invention relates to compositions for the photodynamic control of micro-organisms wherein the compositions comprise a photosensitiser which comprises a dyestuff and produces singlet oxygen when irradiated by means of light for the disinfection and sterilization of materials, commodities and surfaces contaminated with one or more species of micro-organisms including bacteria, fungi, algae, yeasts, bacterial spores and fungal spores.

Related Art

Ancient Egyptian and Indian cultures are reputed to have used coumarin based plant dyes and sunlight to treat skin diseases but it was not until the invention of modern dyes that phototherapies really developed.

The microbiocidal activity of dyes, such as sulfonamidochrysoidine, (Prontosil) was first observed about 80 years ago and led to the discovery of the sulfonamide antibacterials. The selective binding properties of certain dyes were also exploited subsequently in photodynamic therapy (PDT) whereby the particular dye was administered and became bound to selected sites; high intensity light was then applied and cells that contained the bound dye were killed. The mechanism of the antimicrobial action was pronounced to be due to the formation of singlet oxygen because the addition of singlet oxygen traps completely negated the effect. Over the last two decades, photodynamic therapy has become approved in many countries for treatment of cancer and other diseases using non-toxic dyes and visible light wherein the dye acts as a photosensitiser generating a long-lived triplet state that reacts with molecular oxygen to form reactive oxygen species including singlet oxygen, superoxide and radicals.

Quite early in the development of PDT it was found that microorganisms could be killed using a similar combination of photosensitising dyes and light in vitro leading to the control of bacteria, fungi and viruses. This has allowed a better understanding of the molecular processes to be determined and the transfer of this technology to infection control in a strategy referred to as photodynamic inactivation (PDI) particularly of topical conditions. As in the application of traditional chemical microbiocides, vegetative cells were much more susceptible than spores which were found to be resistant to many of the photosensitisers that were commonly used.

A number of methods using photodynamic technology have been used in the past. For example Hamblin (2005) describes the photodynamic inactivation of Bacillus spores mediated by phenothiazine dyes. Other known photosensitisers such as Rose Bengal and porphyrins were only effective against vegetative cells of various Bacillus species. Kill rates of the order of 99.999% were reported but required incubation/contact times of several hours. Candida albicans was also found to be less sensitive to PDI than either Gram positive or negative bacteria; this may be attributed to the greater size of the fungal cells, that being about 20 to 30 time larger.

Phthalocyanines are ‘second generation’ photosensitisers that have two important limitations, that being, aggregation and low water solubility both of which are modulated by variation in substitution. Perfluorination and sulfonation both result in increased water solubility. Zinc and aluminum phthalocyanines are preferred over paramagnetic analogues because of their higher triplet state half-life and yields and singlet oxygen yields.

Nyokong (2007) found that metallophthalocyanine complexes containing diamagnetic metals such as zinc and aluminum were efficient photosensitisers and effective for photocatalytic applications as they generate high triplet state quantum yields and long triplet lifetimes as well as being thermally and chemically stable. Zinc phthalocyanine complexes, in particular, showed higher singlet oxygen quantum yields and found to be more stable towards degradation than its substituted derivatives.

Despite the very high efficiency shown by the above mentioned phthalocyanines, in particular by cationic phthalocyanines, against the majority of micro-organisms, Gram negative bacteria still remain more difficult to inactivate. Even with the most active compounds described above, photoinactivation rates for Gram negative bacteria are at least one order of magnitude lower when compared to the inactivation of Gram positive bacteria and yeast, by using the same photosensitizer and the same experimental conditions.

Photophysical properties of phthalocyanines are affected by substituents, for example zinc phthalocyanine with quaternary ammonium or sulfamido substituents and aluminum phthalocyanine with an ureido substituent were particularly effective in a three hour challenge test against both Gram-negative and Gram-positive bacteria. Wilson (1997) found that the concentration (3-12 ppm) of a sulfonated aluminum phthalocyanine and light dose from a 9 mW laser diode affected kill rate whereas the growth phase of the epidemic methicillin-resistant Staphylococcus aureus (EMRSA) organism did not. The period of exposure to a 35 mW HeNe laser had a greater effect on kill rate than the concentration of a phenothiazine dye or the pre-irradiation contact time—even 15 s was found to be sufficient with a low (ppm) concentration.

Water solubility is an essential requirement for PDT/PDI but aggregation often occurs and such aggregation increases with increasing lipophilicity leading to lower efficacy. U.S. Patent Publication No. 2011/0052817 describes a process for the deposition of water-insoluble aluminum-phthalocyanine dyes from aqueous media using transient water-soluble polyethylene glycol adducts. However, when a solution of the above complex was used to dye a polystyrene sheet and the dyed sheet compared with an undyed sheet it was found to show some residual microbiocidal activity.

Spore formation is a sophisticated mechanism by which some Gram positive bacteria, such as Bacillus anthracis and Bacillus cereus, survive conditions of external stress and nutrient deprivation by producing a multi-layered protective capsule enclosing their dehydrated and condensed genomic DNA. When such bacterial spores encounter a favourable environment, germination can take place, enabling the bacteria to reproduce and, in the case of pathogenic species, cause disease. Bacterial spores possess a coat and membrane structure that is highly impermeable to most molecules that could be toxic to the dormant bacteria. Therefore, spores are highly resistant to damage by heat, radiation, and many of the commonly employed anti-bacterial agents, and can usually only be destroyed by some severe chemical procedures including oxidizing vapors such as peracetic acid, chlorine dioxide and ozone.

Moir (2002) provides information that although the precise molecular details of spore germination have not been fully identified the process involves membrane permeability changes, ion fluxes and activation of enzymes that degrade the outer layers of the spore. Components required for germination include receptor proteins, ion transporters and cortex lytic enzymes; the germinant traverses the outer layers to interact with receptors in the inner membranes in order to initiate the cascade of germination processes.

Collins (2006) described various oxidation based approaches to deactivate spores including use of chlorine dioxide, vaporized hydrogen peroxide and ozone, however peroxides alone rupture the disulfide cross-links but only slowly. The sporicidal action of peroxide can be significantly enhanced by the presence of cationic surfactants and metal complexes and use of organic peroxides such as tert-butylhydroperoxide.

In view of the above said, there is a need for novel pharmaceutical compositions and/or delivery systems, having photodynamic enhanced properties and an increased efficiency, especially against the specific pathologies caused by one or more species of micro-organisms including bacteria, fungi, algae, yeasts, bacterial spores and fungal spores. Further, there is need to avoid any undesired side effects by lowering the dosage of the phthalocyanine photosensitizer, while retaining a high photoinactivation efficiency.

### SUMMARY OF THE INVENTION

The present invention provides methods for the use of photosensitizer compositions to destroy microorganisms including bacteria, fungi, algae, viruses, yeast, bacterial spores and fungal spores. Methods of the present invention are useful in the treatment and prevention of infectious diseases caused by pathogenic microorganisms in humans and animals.

In one aspect, the present invention provides for an aqueous composition (PurePurge) comprising zinc or aluminum phthalocyanine, a carboxylic acid C₆ to C₁₁ and/or hydroxy-acid C₂ to C₆ either as the free acid or as its alkali metal salt; dimethyl sulfoxide and optionally a biological dye selected from oxazine dyes, thiazine dyes and/or triarylmethyl containing dyes. In a preferred aqueous composition the components include zinc phthalocyanine in an amount ranging from about 0.03% wt/vol to about 0.5% wt/vol, sodium octanoate in an amount ranging from about 0.01% wt/vol to about 0.5% wt/vol, a biological dye in an amount ranging from about 0.1% wt/vol to about 0.5% wt/vol, dimethyl sulfoxide in an amount from about 20% wt/vol to about 35% wt/vol and lactic acid in an amount ranging from about 0.20% wt/vol to about 1.5% wt/vol.

The biological dye, if included, may include but is not limited to Benzo[a]phenoxazin-7-ium, 5-amino-9-(diethylamino)-, sulfate (Nile Blue); 3,7-Bis(dimethylamino)phenothiazin-5-ium chloride (methylene blue or methylthioninium chloride); 8-Dimethylamino-2,3-benzophenoxazine hemi(zinc chloride) salt (Meldola Blue); Phenoxazin-5-ium, 3-amino-7-(diethylamino)-2-methyl-, chlorozincate (Brilliant Cresyl Blue); Phenothiazin-5-ium, 3-amino-7-(dimethylamino)-2-methyl, chloride (1:1) (Toluidine Blue); [7-(dimethylamino)-4-nitrophenothiazin-3-ylidene]-dimethylazanium chloride (Methylene Green); Methanaminium, N-[4-[[4-(dimethylamino)phenyl]phenylmethylene]-2,5-cyclohexadien-1-ylidene]-N-methyl-, chloride (1:1) (Victoria Green,); Ethanaminium, N-[4-[[4-(diethylamino) phenyl]phenylmethylene]-2,5-cyclohexadien-1-ylidene]-N-ethyl-, sulfate (Brilliant Green); Methanaminium, N-[4-[[4-(dimethylamino) phenyl]phenylmethylene]-2,5-cyclohexadien-1-ylidene]-N-methyl-, chloride (Malachite Green); 4-[(4-Aminophenyl)-(4-imino-1-cyclohexa-2,5-dienylidene)methyl]aniline hydrochloride (Fuschine) and 4,4′-[(4-imino-2,5-cyclohexadien-1-ylidene)methylene]bisbenzenamine monohydrochloride (Basic Fuschine).

Preferred aqueous composition for PurePurge is setforth below:

| | CAS | |
|---|---|---|
| Ingredients | number | % Wt./Vol. |
| Zinc | 14320-04-8 | 0.046 |
| Phthalocyanine | | |
| Sodium Octanoate | 1984-06-1 | 0.025 |
| Lactic Acid | 50-21-5 | 0.5 |
| Basic Fuchsine | 569-61-9 | 0.125 |
| Dimethyl sulfoxide | 67-68-5 | 27.5 |

In another aspect, the present invention provides for a composition including about a 2% solution of PurePurge comprising zinc phthalocyanine hydroxide as describe above and the 2% solution is mixed with hydrogen peroxide or tert-butylhydroperoxide, and optionally at least one cationic microbiocides, such as, quaternary ammonium and/or quaternary biguanidine compounds.

The cationic microbiocides of the present invention may be selected from among (a) guanidine salts and (b) positive non-metallic salts, preferably quaternary ammonium salts.

The guanidine salts of the present invention may be guanidine salts per se, biguanidinium salts, guanide salts, biguanidine salts or biguanide salts, and all the above are standing for the same molecules.

The guanidine salts of the present invention may be selected from among the following salts, however they are not restricted thereto: chlorhexidine digluconate, dihydrochloride and diacetate; hexamethylenebis(ethylhexyl)biguanide dihydrochloride; oxocyclohexadienylideneaminoguanidine thiosemicarbazone; bis(chlorophenylamidino)piperazinedicarboxami dine dihydrochloride and polyhexamethylenebiguanidine hydrochloride.

The quaternary ammonium salts of the present invention may be selected from among the following salts, however they are not restricted thereto: quaternary salts containing either or both aliphatic or aromatic moieties; aliphatic groups including alkoxy groups which may contain from one to 30 carbon atoms in linear or branched arrangements; alicyclic groups which may be cyclohexyl and its alkylated and alkoxylated derivatives. The preferred quaternary ammonium salt of the present invention is a member selected from the group consisting of alkylbenzyldimethylammonium chloride, alkyl(C₁₂-₁₆)dimethylbenzylammonium chloride and any other cationic surfactants, preferably with an aromatic moiety selected among but not restricted to benzylhexyldimethylammonium chloride, benzyloctyldimethylammonium chloride, benzyldecyldimethylammonium chloride, benzyldodecyldimethylammonium chloride, benzyltetradecyldimethylammonium chloride and, benzyloctadecyldimethylammonium chloride.

Optionally, the compositions may further comprise non-ionic surfactants to enhance microbiocides, such as an alkylpolyglucoside, an alkylethoxylate; a C₉-C₁₀alkyltetraglucoside, a C₉-C₁₁alkylhexaethoxylate, or a C₉₋₁₁ alcoholethoxylate.

Importantly, the present invention provides for the use of water-soluble phthalocyanine compounds in the presence of oxygen and water and on irradiation with light, a particularly good action against micro-organisms, as a result of photodeactivation.

Another aspect of the present invention provides for a kit for cleansing a surface, the kit including the antimicrobial compositions as described herein and at least one applicator for applying the composition. Preferably, the applicator includes an absorbent material, such as a textile either natural or synthetic, and the antimicrobial composition absorbed therein.

A further aspect of the present invention relates to novel, antimicrobial concentrate compositions, as described above, that are capable of being diluted with a major proportion of water or other aqueous based liquid to form a use solution. A still further aspect of the present invention is an aqueous antimicrobial use solution which is particularly suited for “in-place” cleaning applications. Yet another aspect of the present invention is a process of employing the composition of the present invention in the reduction of microbial populations on various contaminated surfaces. Another aspect of the present invention is a method of employing the composition of the present invention in the reduction of microbial populations on various process facilities or equipment as well as other surfaces.

The present invention thus relates to a process for combating micro-organisms in or on organic or inorganic substrates and for protecting the said substrates against attack by micro-organisms and comprises treating the substrates with compositions of the present invention in the presence of oxygen and water and while irradiating with visible, ultraviolet and/or infrared light.

Other aspects and advantages of the invention will be more fully apparent from the ensuing disclosure and appended claims

### BRIEF DESCRIPTION OF THE FIGURE

FIG. 1 summarizes additional test data showing different concentrations and testing organisms for the Meditex and PurePurge formulas.

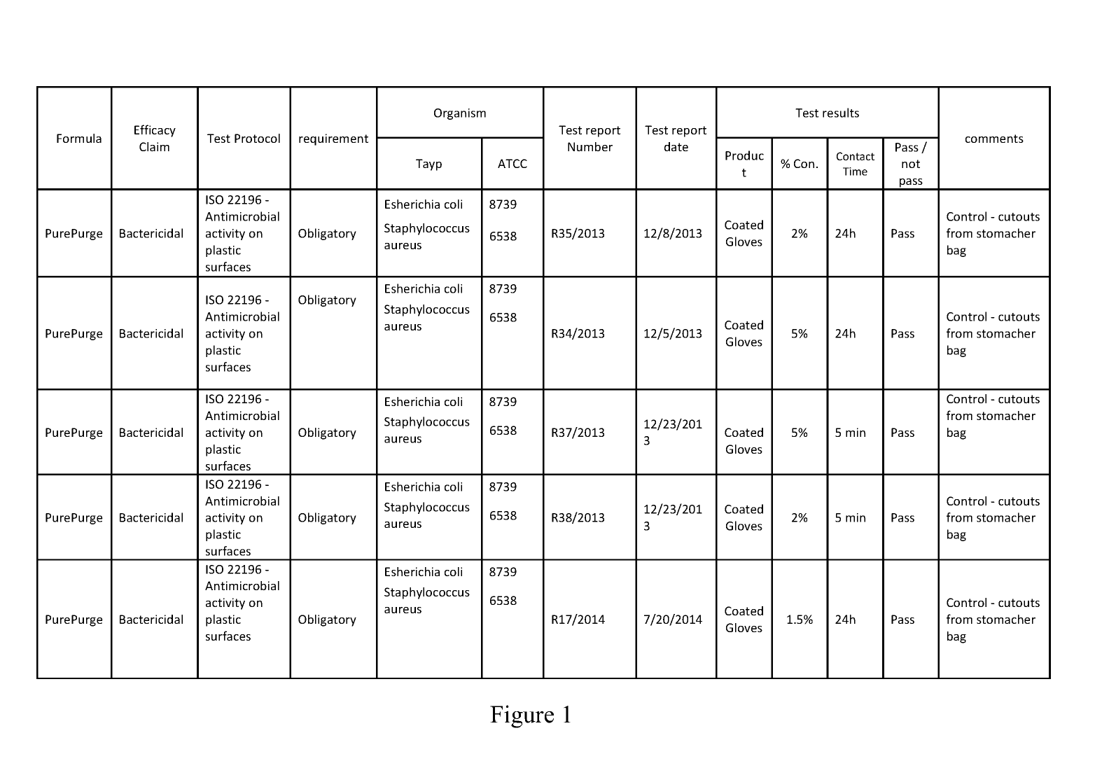

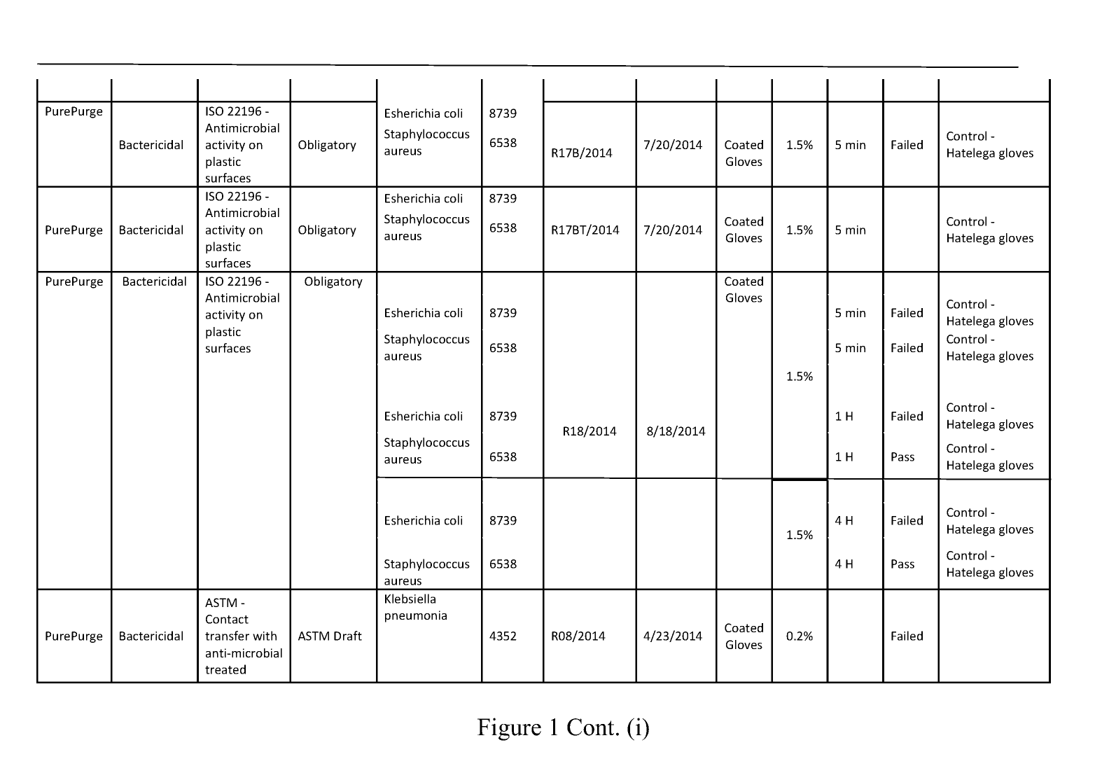

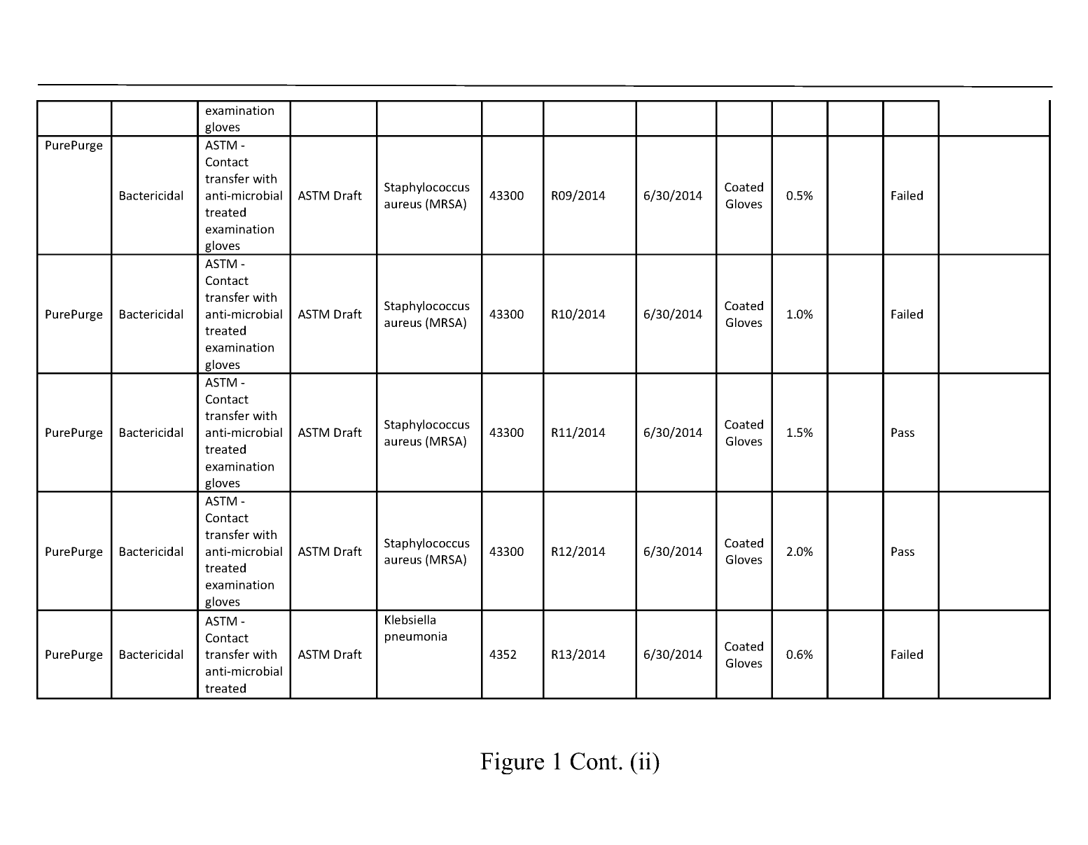

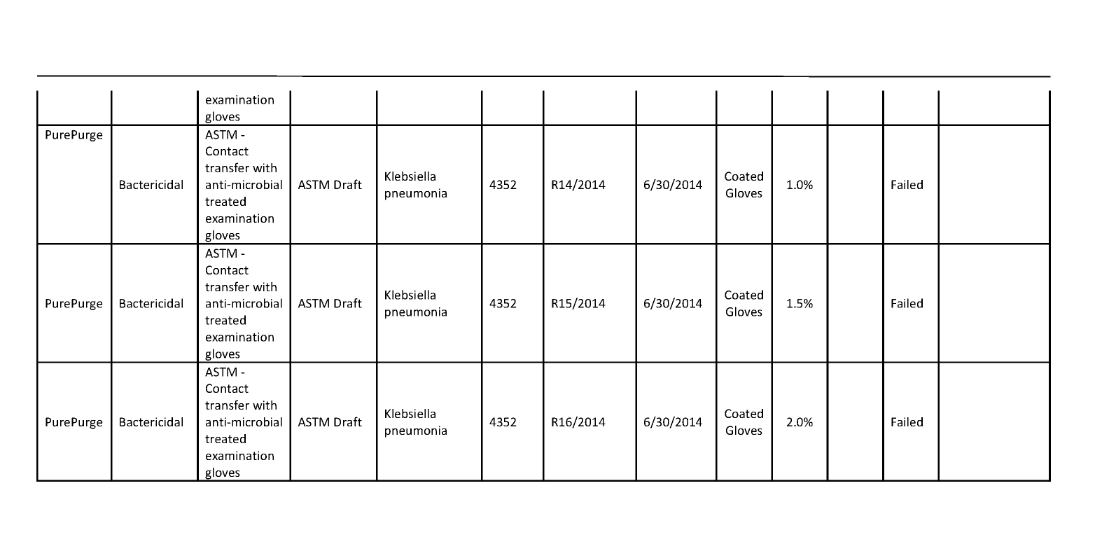

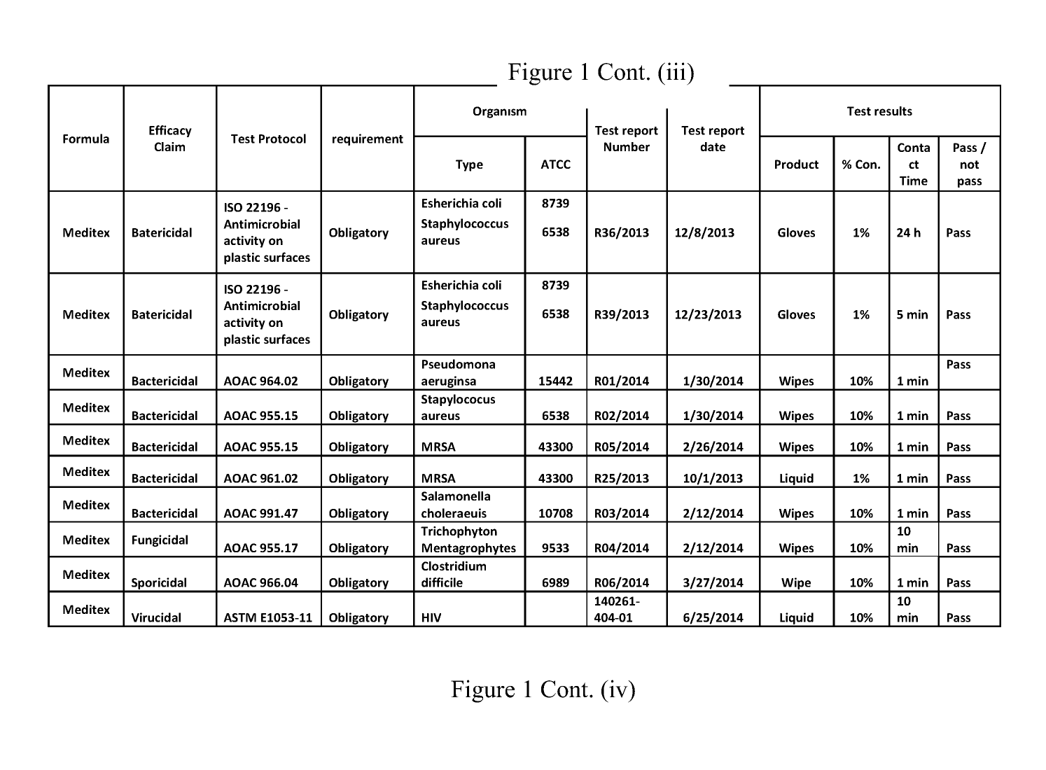

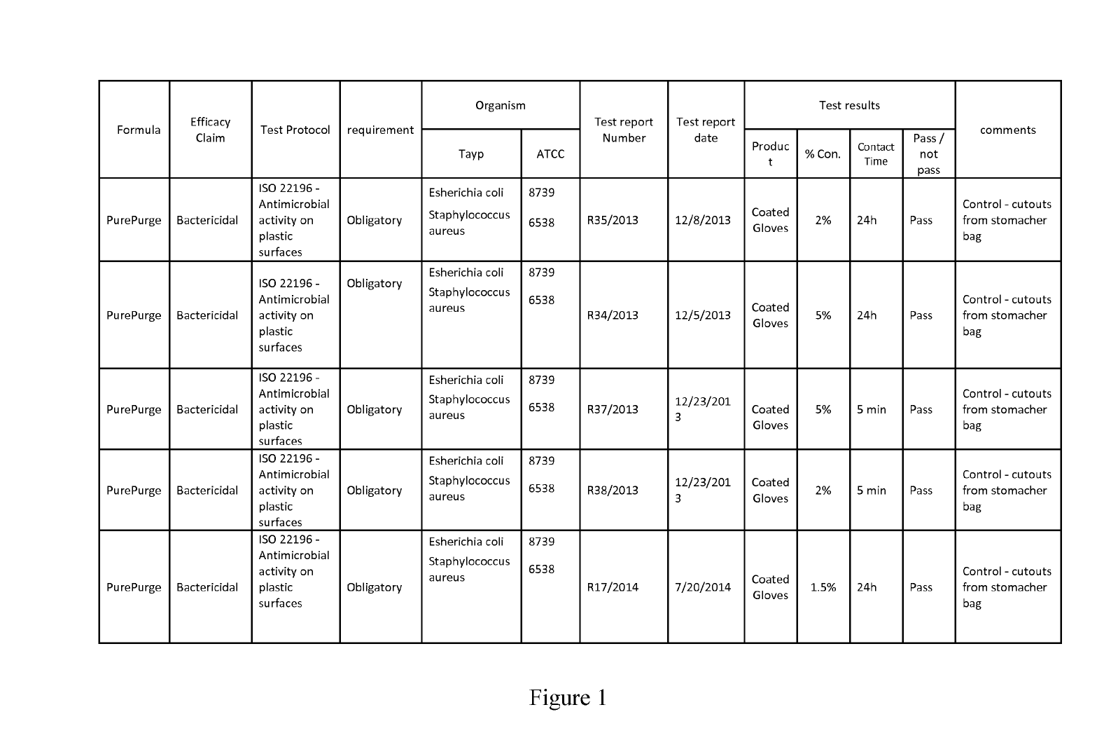

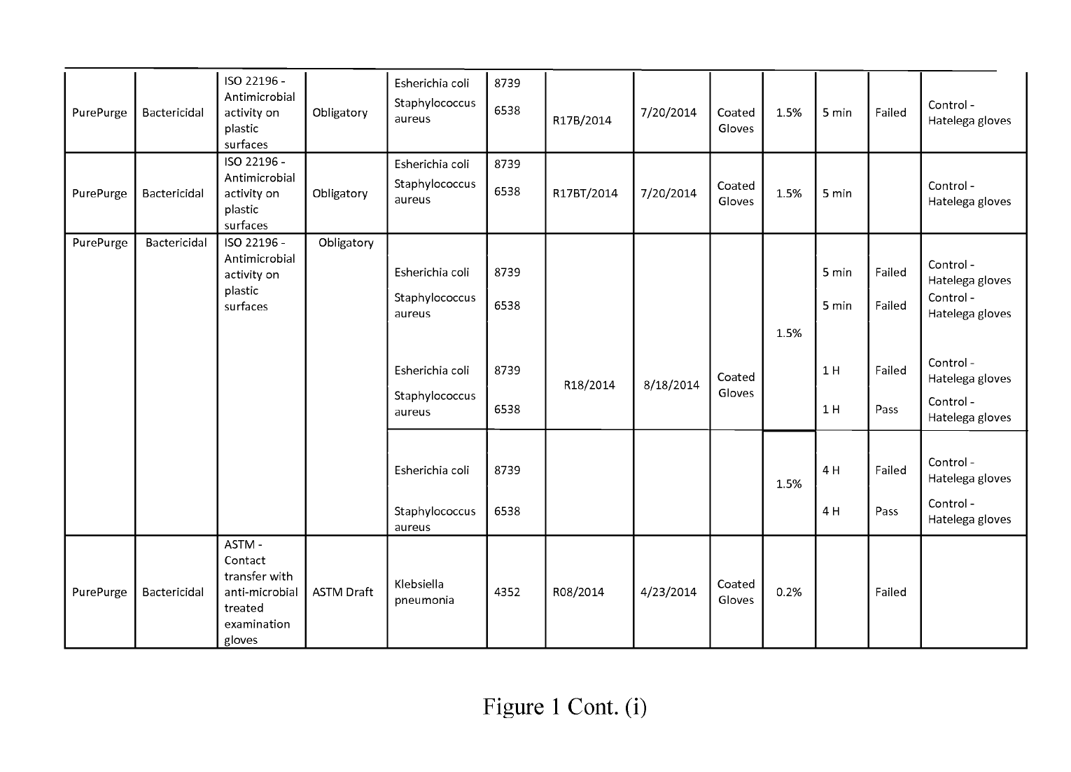

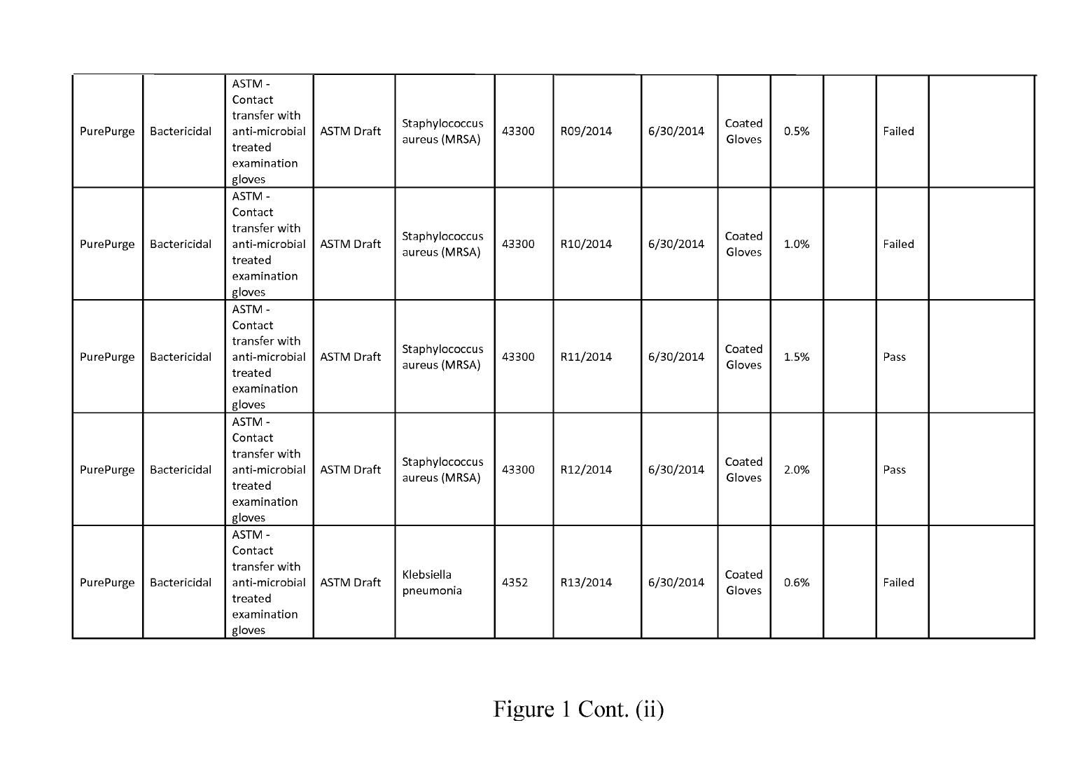

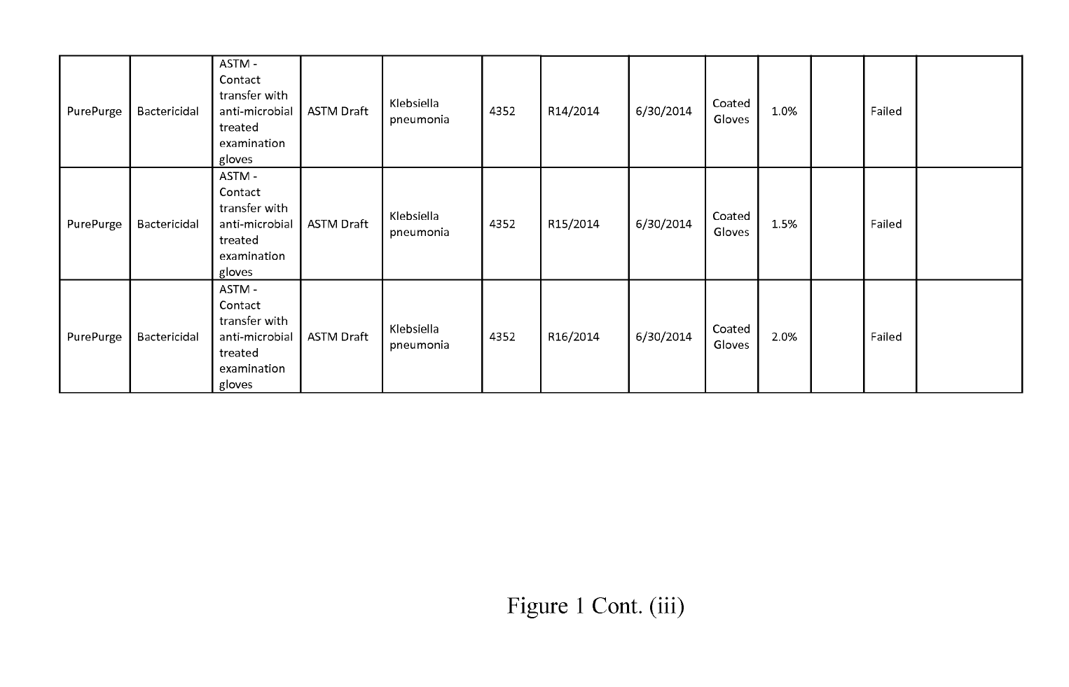

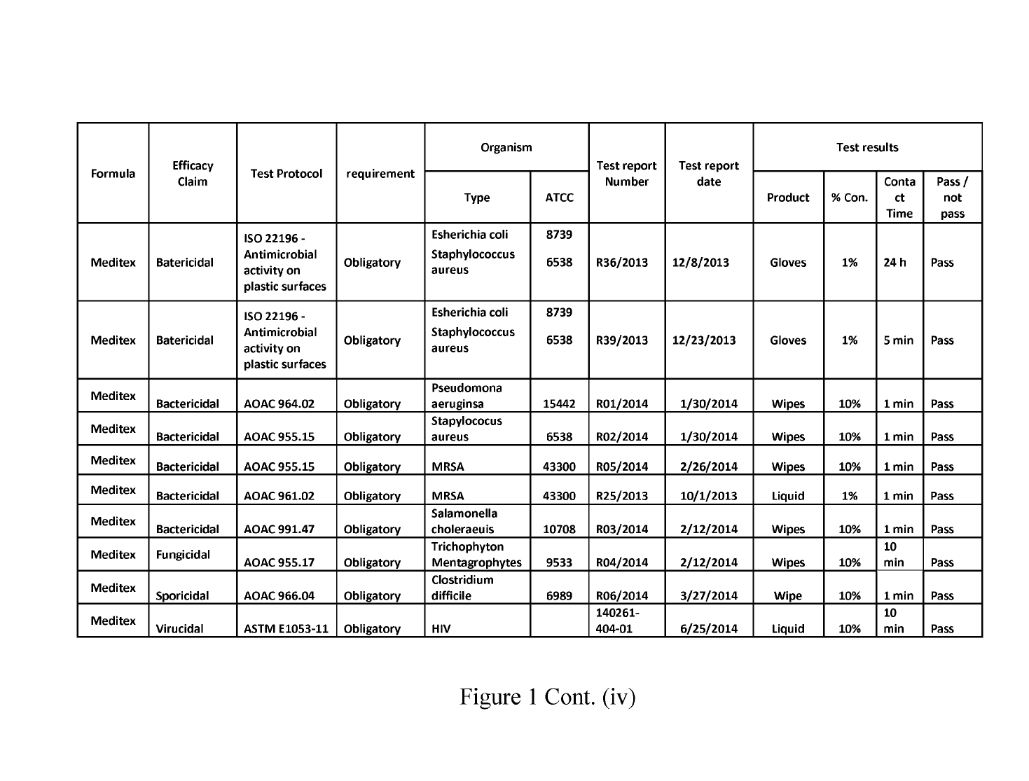

### DETAILED DESCRIPTION OF THE INVENTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

As used herein and in the claims, the term “comprising” is inclusive or open-ended and encompasses the more restrictive terms “consisting essentially of” and “consisting of.”

It must be noted that, as used in this specification and the appended claims, the singular forms “a,” “an” and “the” include plural referents unless the content clearly dictates otherwise. Thus, for example, reference to a “surfactant” includes two or more such surfactants.

In the application, effective amounts are generally those amounts listed as the ranges or levels of ingredients in the descriptions, which follow hereto. Unless otherwise stated, amounts listed in percentage (“%'s”) are in weight percent (based on 100% active) of the cleaning composition.

The antimicrobial cleaning preparation of the present invention may be used for the disinfection and sterilization of materials, commodities and surfaces contaminated with one or more species of micro-organisms including Gram negative bacteria and Gram positive bacteria and are also able to provide residual antibacterial effectiveness against such microorganisms. The components of the present invention may be used in sterilizing compositions for killing and rendering spores lifeless and also may affect the necromass so formed in such a way that it becomes easily removable by water rinsing hence reducing the likelihood of biofilm formation.

The present invention provides for antimicrobial compositions for disinfecting, compositions for use in hygiene (disinfecting), compositions for use in hygiene (sterilizing), disinfectants, anti-bacterial preparations, anti-bacterial compositions for medical use, detergents for medical use, detergents for medical use having anti-bacterial properties, disinfectants for hygiene purposes, disinfectants for medical and veterinary use, disinfectants for hygiene purposes or for medical and veterinary use having anti-bacterial properties, Bactericidal preparations, Fungicidal preparations, Tuberculocidal preparations, Sporicidal preparations, Biocides, chemicals having antimicrobial properties (medical or veterinary), cleaning and sanitizing solutions and compositions for medical use, rinse and drying aids for use in medical washing applications, chemicals for use in cleansing, disinfecting and/or decontamination applications in the medical field, medical scrubs (sterilizing or disinfecting, scrubs preparations) for medical use, surface hygiene products, medicated anti-bacterial washes, anti-bacterial cleansers.

The phthalocyanine compounds used in the present invention require the presence of oxygen and water and also irradiation by visible, ultraviolet and/or infrared light in order to develop their antimicrobial activity. The process is therefore generally carried out in aqueous solutions or on damp substrates and atmospheric oxygen normally serves as the source of oxygen.

Illumination can be by an artificial light source which supplies light in the infrared and/or visible range, or alternatively by sunlight. A good effect is achieved, for example, by light in the range between about 600 and 2,500 nm. Thus, for example, irradiation can be by means of a commercially available filament lamp or by means of an infrared lamp with a λₘₐₓ at about 1,200 to 1,600 nm. The intensity of illumination can vary between wide limits. It depends on the concentration of the active substance and on the nature of the substrate and on the substances additionally present which have an influence on the luminous efficiency. As a further parameter, the exposure time can be varied, i.e. for the same effect, a longer exposure is required at a lower light intensity than at a higher intensity. In general, depending on the field of application, exposure times of a few minutes up to several hours are possible.

If the process is carried out in an aqueous bath (for example disinfecting of textiles), either the irradiation with light can be carried out direct in the treatment bath, by means of an artificial light source located within or outside the said bath, or the substrates, in the damp state, can subsequently either also be illuminated by an artificial light source or exposed to sunlight.

In a further option of the present invention the preparation comprises conventional auxiliary substances for cleaning including fragrances, aromatics, dyestuffs, foam inhibitors, viscosity adjusters and agents for regulating the pH. Such a fragrance material may be any commercial material such as: SAFA 30472 of Parfex S.A., in any desired amount such as 0.1 wt % to 5 wt %. Preferred pH agents include sodium hydroxide and alkanolamines.

Viscosity agents may be used to retain components dispersed in the composition. Examples of suitable agents include silicas, vegetable gums such as xanthans, polymeric gelling agents such as polymers containing carboxyl groups.

As discussed above, the compositions of the present invention are useful in the cleaning and reduction of microbial population of various surfaces including floors, counters, furniture, architectural surfaces, porous surfaces (e.g., textiles, wall paper, carpeting, etc.), medical tools and equipment, food service wares, skin, animal enclosures, feeding stations, veterinarian surgical or examination areas, etc.

Preferably, the compositions of the present invention possess several properties in addition to antimicrobial efficacy. The compositions are preferably no rinse after application, and have residual antimicrobial activity. Residual activity implies a film of sanitizing material which will continue to have antimicrobial effect if the treated surface is contaminated by microorganisms during a period after application of the composition.

A method of reducing microbial population of surfaces comprises the following steps. The composition of the present invention, being a solution formulated for the specific surface and concentration, is introduced onto the surface at a temperature in the range of about 0 to 100° C., and more preferably from about 10 to 25° C. After introduction of the composition, the solution is allowed to remain on the surface for a time sufficient to be effective in reducing the microbial population of the surface (i.e., to kill undesirable microbes) in the presence of an illumination source as described above. After sufficient time for microbial reduction, the use solution is removed. Preferably, the compositions of the invention provide greater than a 99% reduction (2-log order reduction) and more preferably greater than a 99.99% reduction (4-log order reduction) in such microbial population within 5 to 30 minutes at the temperature of treatment.

The practice of the invention is illustrated by the following non-limiting examples.

### EXAMPLE 1

Meditex compositions used as a solution for wipes.

Solutions (2% aqueous) of the PurePurge were mixed with hydrogen peroxide, tert-butylhydroperoxide, with and without quaternary ammonium/biguanidine disinfectants and investigated for their sporicidal activity against Clostridium difficile spores (AOAC 961.02)

Compositions & Microbiological Results for the Meditex compositions shown in the following Testing examples. Values of ingredient amounts in the below table are percentages (by weight).

| Ingredient | Solution 1 | Solution 2 | Solution 3 | Solution 4 | Solution 5 |
|---|---|---|---|---|---|
| Al-pc-PEG | 100 | 95 | | | |
| (2%)/98% | | | | | |
| water | | | | | |
| PurePurge | | | 96 | 90 | 93 |
| (2%)/water | | | | | |
| (98%) | | | | | |
| ADBAC | | | 1.5 | 1.5 | 1.5 |
| PHMB | | | 2.5 | 2.5 | 2.5 |
| HPx | | | | 6 | |
| TBHPx | | | | | 3 |
| QAC/BG | | 5 | | | |
| mix | | | | | |
| Irradn Test | 2.00 | | | | |
| LgR (13704) | <1.5 | <1.5 | 2.8 | | |
| AOAC | fail | fail | | pass | pass |
| EN13704 | | | | low | low |

Abbreviations and footnotes:
Al-pc-PEG: aqueous solution prepared from aluminum phthalocyanine chloride (CAS RN=14154-42-8) with methoxypolyethylene glycol (MW 2000Da., CAS RN=9004-74-4) according to US Patent 2011/0052817; 2% aqueous solution.
ADBAC Alkyl(C12-16)dimethylbenzylammonium chloride (CAS RN=68424-85-1) 50% aqueous solution.
PHMB Polyhexamethylenebiguanidine hydrochloride (CAS RN=27083-27-8) 20%aqueous solution.
HPx Hydrogen peroxide (CAS RN=7722-84-1) 30% aqueous solution
TBHPx tert-Butylhydroperoxide (CAS RN=75-91-2) 70% aqueous solution
QAC/BG commercially available mixture of quaternary ammonium compounds and biguanidine microbiocides in aqueous isopropanol (4% total biocides)
Irradn Test Log reduction value achieved in custom designed challenge test using Staphylococcus aureus and bright light illumination with 24 hour contact. See also Hamblin 2005 for validated test protocol for light activated antimicrobial efficacy.
LgR Log₁₀ reduction achieved in EN13704 and AOAC2008.05 tests with 1 & 3 minute contact times and AOAC 961.02 test of disinfectant efficacy with 1 minute contact time.

The results from these studies show a significantly enhanced sporicidal activity: the microbiocidal activity is against spores rather than vegetative cells and follows very short contact times of one to three minutes compared to hours for literature reports of photodynamic activation alone. Surprisingly, the microbiocidal particularly the sporicidal efficacy of aqueous solutions of aluminum and/or zinc phthalocyanine complexes can be considerably and significantly enhanced by combination with solutions of quaternary ammonium, biguanidine microbiocides and hydroperoxides.

### EXAMPLE 2

| | | | Active | |
|---|---|---|---|---|
| | | | component | 10% |
| Component Name | Symbol | CAS Number | concentration | 4.SUN.22101.1 |
| t-buthlydroperoxide | tPHBx | 75-91-2 | 70% | 1.500 |
| Alkyl(C12-16)dimethylbenzyl | BAC | 68424-85-1 | 50% | 0.750 |
| ammonium chloride | | | | |
| Polyhexamethylenebiguanide | PHMB | 27083-27-8 | 20% | 1.250 |
| hydrochloride | | | | |
| Perfume - SAFA | | | | 0.100 |
| PurePurge 2% | | | | 0.0050 |

| | |
|---|---|
| Water | 96.395 |
| TOTAL | 100.000 |

The above described MEDITEX solution, at different concentrations, was tested on several different types of bacterium or fungi and the results are shown below.

1. Fungicidal activity for reference strain of Trichophyton mentagrophytes ATCC #9533.

| Name | Concentration |
|---|---|
| MEDITEX wipes | 10% |

2. Reference:

Test Method: According to AOAC international, 2000, Official Methods of Analysis. Volume 1, Chapter 6, 955.17 Fungicidal Activity of Disinfectants.

3. Media:
a) Culture Media:
Glucose Broth
Glucose Broth with 0.07% lecithin, 0.5% polysorbate 80
Glucose agar
b) Test organism: Trichophyton mentagrophytes ATCC #9533

4. Introduction:

This report details the evaluation of procedures for the evaluation of Disinfectant for fungicidal efficacy. The product was tested against Trichophyton mentagrophytes ATCC #9533, following the procedures outlined in the AOAC Official Methods of Analysis, Fungicidal Activity of Disinfectants (955.17).

5. Procedures:

Inoculum Preparation:

Trichophyton mentagrophytes ATCC #9533 was transferred to 5 plates of glucose agar and incubated at 25-30° C. for 10-15 days. After incubation, the mycelial mats were removed from the agar surface using a sterile spatula, transferred to a sterile tissue grinder and macerated using 25 ml of phosphate buffer. The suspension was filtered through a sterile funnel containing moist cotton and the suspension was standardized with phosphate buffer to contain ˜10⁶ conidia/ml. A standard plate count was performed on the conidial suspension to verify the titer of the test organism.

Test performance:

Five ml of each of the test solutions were placed in sixty 25×150 mm test tubes and the tubes were placed in a 20+1° C. water bath. Using a calibrated micropipetor, 0.5 ml of conidial suspension was placed in the first tube of test solution, shaken, and immediately replaced in the water bath. At 30 second intervals, 0.5 ml of the conidial suspension was added to the second tube. This was repeated at 30 second intervals until all tubes were inoculated. After 10 minute, a sample from each tube was removed with a 4 mm loop and placed into 20 ml of glucose broth. The tubes were incubated at 27-29° C. for 10 days and then was examined for growth of the challenge organism.

Phenol Resistance:

The phenol resistance of the test culture was determined according to the phenol dilutions of 1:60 and 1:70. A 5% stock solution of the phenol (1:20) was diluted further to make the needed dilutions. Five milliliter aliquots of each dilution were placed into sterile test tubes and allowed to equilibrate in a 20+0.5° C. water bath. An additional tube was prepared and the thermometer was placed in that tube to show when the phenol dilutions had equilibrated to the test temperature. One half milliliter of the conidial suspension was added to each tube at 30 second intervals. The tubes were gently agitated to distribute the culture and replaced in the water bath. The exposure times were 5, 10, and 15 minutes. After the appropriate exposure time, a loop full (4 mm loop bent at a 300 angle) was removed from the assay tube and transferred to a tube of LETH. The tubes of LETH were incubated at 27-29° C. for 4 days. Growth was reported as being either positive or negative for the challenge organism.

Neutralization Verification:

Six tubes without growth were selected, and to each tube was added appropriate amount of test strain culture to deliver 5-100 CFU/tube. The tubes were incubated and were checked for growth/no growth. Acceptance criteria: growth in all tubes. The number of the CFU that was added to each tube was confirmed by pour plate method.

Growth Promotion of Media:

According to laboratory quality assurance practice.

Sterility Controls:

Tow tubes of glucose broth were included with the test as a media control. All tubes were incubated with the test in order to confirm sterility of the recovery media used in the test.

6. Results:

The titer of Trichophyton mentagrophytes was 5.7×10⁶ conidia/ml.

The conidia must survive a 10 minute exposure to phenol dilution 1:70, but not 1:60. The test culture survived a 10 minute exposure to both phenol dilutions, providing a more severe challenge for the test. Neutralization efficacy: all media tubes demonstrated growth by 4 days.

7. Conclusion:

According to AOAC Official Methods of Analysis, Fungicidal Activity of Disinfectants (955.17) the product: MEDITEX wipes 10%, at 10 min contact time, possesses satisfactory fungicidal activity for reference strain of Trichophyton mentagrophytes ATCC #9533.

### EXAMPLE 3

Disinfection against Staphylococcus aureus ATCC 43300 (MRSA)

Identification of test Sample

| | |
|---|---|
| Name of product | MEDITEX wipes |
| Active substance(s) and its (their) | 10% concentration(s) |
| Application | Disinfection |

2. Reference:

AOAC Official Method 955.15. Testing Disinfectants against staphylococcus aureus use dilution method.

3. Purpose:

To determine the maximum dilutions, effective for practical disinfectant.

4. Media:
Culture Media:
Nutrient broth
Synthetic Broth
Letheen broth
Nutrient agar
TSA agar
Baird Parker Agar

5. Test organisms:

Staphylococcus aureus ATCC 43300 (MRSA)

6. Apparatus:
Carriers: Polished stainless steel cylinders (6×8×1 mm)
Glassware—Disposable sterile volumetric pipettes, flasks, borosilicate glass tubes.
Petri dishes.
Inoculating loop.
pH meter.
Capillary pipettes—0.1 ml, graduated to 0.01 ml.
Microscope slides.
Water bath
Sonicator
Glass beads.
Incubator.
Dacron swab
Sterile absorbent pads
Spectrophotometer

7. Carrier preparation:

In accordance with AOAC 955.15

8. Test culture preparation: In accordance with AOAC 955.15

9. Disinfectant sample preparation:

The ready to use disinfectant was dispensed into 25×10 mm test tubes, 10 ml in each tub, one tube per carrier. The tubes were placed in equilibrated water bath for 10 min.

10. Operating Technique:

Each of the stainless steel cylinders (“carriers”) was transferred to a tube containing the test culture. After 15±2 minutes the carriers were removed from the tubes, the carriers were shaken to remove excess culture and were placed in vertical position on filter paper in petri dishes. The plates were incubated for 40 minutes in 36° C. incubator. After the required drying time, the carriers containing the dried organism film were sequentially transferred from the petri dish to the test tube containing the disinfectant. After the exposure time was completed (1 min) the carriers were sequentially transferred to a liquid subculture medium (Letheen broth) tubes. The subculture tubes containing the carriers were incubated in 36° C. for 48 h. A visually examination was performed for presence or absence of turbidity (growth or no growth)

11. Viability control:

2 dried inoculated carriers were placed into separate tubes containing 10 ml of Letheen broth and the tubes were incubated in 36° C. for 48 h.

12. Verification of the positive carrier

Positive carrier were examined for test organism by inoculation onto TSA agar and Baird Parker agar.

13. Enumeration of the viable bacteria from the carriers

5 inoculated dried carriers were placed in letheen broth tubes and were sonicates for 1 minute in sonication bath. The bacterial count was determined by pour plate method using 10-folds dilutions and TSA agar plates.

14. Neutralization confirmation:
Five negative tubes (with no growth) were inoculated with 0.01 ml of 100-1000 cfu/ml of the test microorganism
The tubes were incubated in 36° C. for 48 h.
The tubes were examined for growth.

15. Acceptance criteria:

No Growth in 57 out of 60 test tubes for the test organism. Growth in each of the neutralization confirmation tubes. Growth in each of the viability control tubes. Mean Log 10 Density: 6.0-7.0. No contamination of non-test organisms in positive tubes.

16. Conclusions:

The disinfectant Meditex wipes 10% conforms to the requirements of AOAC 955.15 for disinfection against Staphylococcus aureus ATCC 43300 (MRSA) at 1 min contact time.

| Tube | 1 | 2 | 3 | 4 | 5 | 6 |
|---|---|---|---|---|---|---|
| 1 | No Growth | No Growth | No Growth | No Growth | No Growth | No Growth |
| 2 | No Growth | No Growth | No Growth | No Growth | No Growth | No Growth |
| 3 | No Growth | No Growth | No Growth | No Growth | No Growth | No Growth |
| 4 | No Growth | No Growth | No Growth | No Growth | No Growth | No Growth |
| 5 | No Growth | No Growth | No Growth | No Growth | No Growth | No Growth |
| 6 | No Growth | No Growth | No Growth | No Growth | No Growth | No Growth |
| 7 | No Growth | No Growth | No Growth | No Growth | No Growth | No Growth |
| 8 | No Growth | No Growth | No Growth | No Growth | No Growth | No Growth |
| 9 | No Growth | No Growth | No Growth | No Growth | No Growth | No Growth |
| 10 | No Growth | No Growth | No Growth | No Growth | No Growth | No Growth |
| Viable bacteria | 2 .4 × 10⁶ | 5.3 × 10⁶ | 4.1 × 10⁶ | 5.1 × 10⁶ | 8.3 × 106 | NA |
| per carrier | | | | | | |
| neutralization | No Growth | No Growth | No Growth | No Growth | No Growth | NA |
| confirmation | | | | | | |
| Viability | Growth | Growth | NA | NA | NA | NA |
| controls | | | | | | |

| | |
|---|---|
| Verification | NA |
| of the | |
| positive carrier | |

### EXAMPLE 4

Importantly the Meditex composition was found effective at a 1% concentration for disinfecting against Staphylococcus aureus ATCC 43300 (MRSA), as shown below: Identification of test Sample

| | |
|---|---|
| Name of product | Meditex |
| Active substance(s) and its (their) | 1% concentration(s) |
| Application | Disinfection |

2. Reference:

AOAC Official Method 961.02. Germicidal Spray Products as Disinfectants. First Action 1961. Final Action 1964.

3. Purpose:

To determine the effectiveness of sprays and pressurized spray products as spot disinfectants for contaminated surfaces.

4. Media:

Culture Media:
Nutrient broth
Bacto Synthetic Broth AOAC
Letheen broth
Trypticase soy agar
Nutrient agar
Mannitol salts agar
Saline solution (0.85% NaCl)
Sterile distilled water

5. Test organisms:

Staphylococcus aureus ATCC 43300 (methicillin resistant)-MRSA

6. Apparatus:
Glassware—Disposable sterile volumetric pipettes, flasks, borosilicate
glass
tubes.
Petri dishes.
Inoculating loop.
pH meter.
Capillary pipettes—0.1 ml, graduated to 0.01 ml.
Microscope slides.
Water bath
Sonicator
Glass beads.
Incubator.
Dacron swab
Sterile absorbent pads
Spectrophotometer

7. Operating Technique:

18-48 hour nutrient broth culture of Staphylococcus aureus was thoroughly shaken. 0.01 ml of test suspension was transferred onto sterile test slide in petri dish and immediately spread over the entire area. The dish was covered immediately. The process was repeated until there were 12 slides (2 of them are for control). All slides were dried at 37° C. for 30-40 min. 10 slides were sprayed for 10 sec. at a distance of 1 ft. Each slide was held for 1 min, drained of excess fluid and was transferred to a tube containing 20 ml of the appropriate subculture medium. The culture was shaken thoroughly. 2 unsprayed slides were transferred to individual subculture tubes (viability controls). The process has been repeated 5 times. All tubes were incubated at 37° C. for 48 h. Results were read as Growth/No Growth.

8. Neutralization confirmation:

3 sprayed slides were transferred to the glucose broth containers for 30 min. Afterwards the slides were taken out and inoculated with 0.01 ml of the inoculum and transferred to letheen broth.

9. Acceptance criteria:

No Growth in 58 out of 60 test tubes for the test organism. Growth in confirmation of the neutralization tubes. No contamination of non-test organisms in positive tubes.

10. Conclusions:

The disinfectant Meditex conforms to the requirements of AOAC 961.02 for disinfection against Staphylococcus aureus ATCC 43300 at 1 min contact time.

### EXAMPLE 5

Purpose: To evaluate efficacy of hard surface disinfectant against Salmonella choleraesuis.

Product:

| Name | Concentration |
|---|---|
| MEDITEX wipes | 10% |

Reference: AOAC Official Method 991.47. Testing Disinfectants Against Salmonella choleraesuis. Hard Surface Carrier Test Method. First Action 1991.

Media:
Culture Media:
Nutrient broth
Bacto Synthetic Broth AOAC
Letheen broth
Trypticase soy agar
Nutrient agar
MacConkey's agar
Phosphate buffer dilution water (PBDW)
Filter-sterilized glucose solution, 10% (weight/volume)

3. Test organism: Salmonella choleraesuis ATCC 10708

4. Apparatus:
Glassware—Disposable sterile volumetric pipettes, flasks, borosilicate
glass tubes.
Petri dishes.
Water Bath, capable of maintaining temperatures of 20° C.±0.5° C.
Test tube racks.
Inoculating loop.
Wire hook.
Thermometer.
Carriers—disposable fire-polished borosilicate glass, 10±1 mm in length, 6±1 mm id, 8±1 mm od.
Dacron swab.
Sterile absorbent pads with dispenser—47 mm diameter cellulose fiber pads.
Sterile filtering system.
Sonicator.
Spectrometer.
pH meter.

5. Operating Technique:

Carrier preparation: In accordance with AOAC 991.47 C(a).

Culture preparation: In accordance with AOAC 991.47 C(d).

Development of standard curve: In accordance with AOAC 991.47 C(c).

6. Culture standardization:

The percent transmittance of the culture that was prepared in accordance with paragraph b) was determined. The culture was adjusted, using synthetic broth, to a concentration of 5-10×10⁹ cfu/ml.

7. Carrier inoculation:

Water was removed aseptically from prepared carriers and 24 ml (1 ml per for each carrier) of standardized culture were added (the carriers were completely submerged). The test tubes were capped and kept at room temperature for 15 min. The carriers were then removed from suspension until 12 carriers were placed in a petri dish with filter paper. Each carrier (after removal of excess medium) was moved to stand separately and upright on a dry section of the filter paper. The inside of the ring of each carrier was dried. The dishes were covered and transported to the incubator with the carriers kept upright. The dishes were dried in the incubator at 37° C. for 40 min. Each carrier was placed in 10 ml of letheen broth and sonicated for 10 min. The process was repeated to obtain 60 carriers for testing. The carrier bacterial load was counted on one cylinder from each plate.

8. Test product preparation:

10 ml of test solution (has been squeezed from wipes) were put into each one of 20 test tubes. The tubes were placed into a water bath at 20±0.5° C. for more than 10 min, until the contents reached the bath water temperature. The process was repeated to obtain 60 tubes for testing.

9. Carrier exposure:

Contaminated and dry carrier was added to 1 test product tube every 30 sec. The timer was started at the first carrier. At 1 minute, carriers were removed every 30 seconds in the order of insertion and transferred to tubes of letheen broth. The tubes were shaken and incubated at 37° C. for 48-54 h. Positive tubes were confirmed for the growth of test organism.

10. Neutralization confirmation:

Negative tube was randomly selected for every 10 tubes tested. They were inoculated with less than 100 cfu/tube (the exact number was determined using the pour plate method). The tubes were incubated at 37° C. for 48-54 h and examined for growth.

11. Acceptance criteria:

Average bacterial count must be 0.5-2.0×10⁶ cfu/dried carrier. <2positives (growth) carriers out of 60 tested. No contamination of non-test organisms in positive tubes. Growth in all neutralization confirmation tubes.

12. Conclusions:

The disinfectant MEDITEX wipes 10% conforms to the requirements of AOAC 991.47 for disinfection against Salmonella choleraesuis at 1 min contact time.

### EXAMPLE 5

This testing was conducted to test the effectiveness of a 2% solution of PurePurge for Evaluating Microbial Contact Transfer with Antimicrobial-Treated Examination Gloves

1. Identification of test Sample

| | |
|---|---|
| Name of product | Nitrile powder free 3.0 Mil White |
| Batch .NQ | RD/14032014/02SG |
| Active substance(s) | coated with 2% PurePurge on external side |
| Application | Disinfection |

2. Reference: Standard Test Method for Evaluating Microbial Contact Transfer with Antimicrobial-Treated Examination Gloves

3. Reagents and Materials
Bovine albumin serum
Letheen broth
Letheen agar
PBS
Stainless steel coupon
TSB

4. Experimental conditions:

| | |
|---|---|
| Test Temperature | 20° C. + 0.5° C. |
| Concentration of test product | NA |
| Contact time | 1 min exposure, 5 min wait, |
| | 1 min exposure again |
| Interfering substance | 5% bovine albumin |
| Neutralization agent | Letheen broth |
| Diluent | Water of standardized hardness |
| Microorganism | Staphylococcus aureus ATCC 43300 |
| Temperature of incubation | 32° C. + 1° C. |

5. Methods

Introduction: This test method is used to measure the ability of an antimicrobial treated examination glove to reduce skin to surface and surface to skin contact transfer of a known population of bacteria

Preparation of Microorganisms Stock Suspension

The microorganisms were recovered twice by suspending in TSB medium and incubation in 37±2° C. for 18±2 h with shaking. After the incubation the microorganisms were centrifuged and washed 3 times with PBS. The microorganisms were resuspended again in PBS containing 5% Bovine albumin serum.

Preparation of Gloves

The gloves were exposed to light for 1 min before beginning of the test.

Principle:

100 μl of the prepared inoculum were placed on the stainless steel coupon and spread for 1 min. The gloves were allowed to contact the stainless steel coupon for 1 min. The gloves were allowed to sit for 5 min at ambient room condition. Afterwards the gloves were allowed to contact with new stainless steel coupon for 1 min. After the exposure period the stainless steel coupon were placed into jar containing 25 ml letheen broth. The jars were vortexed for 2 min and then were sonicated for 5×1 min intervals with a 1 min wait between each interval. The jars were vortexed again for 1 min. The bacterial count was determined by 10 fold serial dilutions up to 10⁻⁶. All dilutions were plated in duplicate. The plates were incubated at 35±2° C. for 48±4 h.

6. Results: summarized in the table below

| | | | | Difference in | |
|---|---|---|---|---|---|
| | | Average | Average | the reduction | |
| | Inoculum | log of | log of | of bacteria | |
| Microorganism | cfu/ml | gloves tested | control | (log) | T test |
| Staphylococcus | 3.8 × 10⁸ | 3.00 | 3.99 | 0.99 | 0.01 |
| aureus | | | | | |
| ATCC 43300 | | | | | |

7. Conclusion: According to Test Method for Evaluating Microbial Contact Transfer with Antimicrobial-Treated Examination Gloves, the product: Nitrile powder free 3.0 Mil White coated with 2% PurePurge on external side showed ability to reduce skin to surface and surface to skin contact transfer of a Staphylococcus aureus ATCC 43300. Notably the same testing was conducted on Klebsiella pneumonia ATCC 4352 but was found not to be effective.

### EXAMPLE 7

The following examples show the effectiveness of the combination of the following components and combined in an aqueous solution to provide a solution with different concentrations of PurePurge formulation.

| | | % |
|---|---|---|
| Ingredients | CAS No | Wt/Vol |
| Zinc Phthalocyanine | 14320-04-8 | 0.046 |
| Sodium Octanoate | 1984-06-1 | 0.025 |
| Methylthioninium chloride | 7220-79-3 | 0.25 |
| Dimethyl sulfoxide | 67-68-5 | 27.5 |

Gloves treated with 2% PurePurge under dirty conditions for both 5 minutes and 24 hours.

1. Reference: ISO 22196:2007(E). Plastics—Measurement of antibacterial activity on plastics surfaces.

2. Introduction:

A defined surface area is treated with the product and after 5 min contaminated with the tested microorganism.

3. The Product:

Product Name
Gloves treated with 2%
PurePurge

4. Acceptance Criteria:

The average U0 is : 6.2×10³≦U0≦2.5×10⁴.

The average Ut ≧6.2×10¹.

5. Test Organisms:

Esherichia coli ATCC 8739

Staphylococcus aureus ATCC 6538

6. Media:
TSB (Tryptic Soy Broth)
PBS (phosphate buffer saline)
TSA (Tryptic Soy Agar) BP (Baird Parker Agar) TBX Medium
Neutralizer:
LB (Modified Letheen Broth)

7. Preparation of the test organism:

An overnight incubated culture of each of the target organisms will be grown in TSB at 37° C. for a minimum of 18 hours. The overnight culture will be adjusted to give a bacterial concentration of 2.5×10⁵ cfu/ml to 10×10⁵ cfu/ml using PBS. A serial dilution of the inoculum will be prepared and plated out on TSA to obtain an initial inoculum count. The plates will be incubated at 37° C. for 24 hours.

8. Test specimens: Flat, 50 mm×50 mm sheets of the Product.

9. Control specimens: Flat, 50 mm×50 mm Cutouts from a stomacher bag. Film: Cutouts from a stomacher bag (40 mm×40 mm)

Interfering substance:

10. Dirty Conditions—3.0 g/l bovine albumin

11. TEST OPERATION:
The tests were performed in triplicate.
9 test specimens were each placed into a separate sterile petri dish.
3 specimens were of the test specimens.
6 specimens were of the control specimens.

After the exposure period, each of the specimens was inoculated with 0.4 ml of the tested microorganism suspension. Each specimen was covered with a piece of film. The film was gently pressed down, so that the test inoculum would spread to the edges. Each petri dish was covered with the lid. 3 of the untreated and inoculated specimens were held for 5 minutes and then the bacteria were recovered. The other petri dishes (3 test and 3 control specimens) were incubated at 35° C. for 5 min.

12. Recovery: the specimens were transferred to individual containers, containing 10 ml of neutralizing broth. The containers were thoroughly shaken. The survival of the microorganisms after incubation was determined by using standard microbiological serial dilutions, pour plate count procedure and selective media plates (TBX for Esherichia coli). The plates were incubated at 37° C. for 24 hours. The number of microorganisms recovered after incubation was calculated.

13. Interfering substance sterility control:

The interfering substance was cultured, incubated and visually examined for growth. The acceptance criterion for this study control is lack of growth.

14. Carriers' sterility control:

Three uninoculated carriers were added to TSB medium, incubated and visually examined for growth. The acceptance criterion for this study control is lack of growth.

15. Results:

| | U0 | Ut | At | R |
|---|---|---|---|---|
| E. coli | 4.17 | 4.36 | 2.66 | 1.70 |
| ATCC 8739 | | | | |
| Staphylococcus | 3.34 | 3.42 | 2.25 | 1.17 |
| aureus | | | | |
| ATCC 6538 | | | | |

Abbreviations:
R—The antibacterial activity.(log reduction).
U0—The average of the common logarithm of the number of viable bacteria, in cells/cm², recovered from the untreated test specimens immediately after inoculation.
UT—The average of the common logarithm of the number of viable bacteria, in cells/cm², recovered from the untreated test specimens after 5 min.
At—The average of the common logarithm of the number of viable bacteria, in cells/cm², recovered from the treated test specimens after 5 min.

| | | |
|---|---|---|
| Control | Control 1 | Control 1 |
| Interfering substance sterility | No Growth | No Growth |
| Control Specimens sterility | No Growth | No Growth |

16. Conclusion:

In reference to ISO 22196, the product Gloves treated with 2% PurePurge, batch No. lab/281013/2, possess bactericidal activity on surfaces under dirty conditions at 5 min for referenced strains of Escherichia coli, Staphylococcus aureus.

Notably, the same conditions were used to test for growth after 24 hours. The following shows the results for 24 hours.

17. Results for 24 hours:

| | U0 | Ut | At | R |
|---|---|---|---|---|
| E. coli | 4.18 | 4.04 | <1 | >4 |
| ATCC 8739 | | | | |
| Staphylococcus | 4.01 | 4.81 | <1 | >4 |
| aureus | | | | |
| ATCC 6538 | | | | |

Abbreviations:
R—The antibacterial activity. (log reduction).

U0—The average of the common logarithm of the number of viable bacteria, in cells/cm², recovered from the untreated test specimens immediately after inoculation.
UT—The average of the common logarithm of the number of viable bacteria, in cells/cm², recovered from the untreated test specimens after 24 h.
At—The average of the common logarithm of the number of viable bacteria, in cells/cm², recovered from the treated test specimens after 24 h.

| | | |
|---|---|---|
| Control | Control 1 | Control 1 |
| Interfering substance sterility | No Growth | No Growth |
| Control Specimens sterility | No Growth | No Growth |

18. Conclusion:

In reference to ISO 22196, the product Gloves treated with 2% PurePurge, batch No lab/281013/2, possess bactericidal activity on surfaces under dirty conditions at 24 h for referenced strains of Escherichia coli, Staphylococcus aureus.

### EXAMPLE 8

Gloves treated with 5% PurePurge under dirty conditions for both 5 minutes and 24 hours.

1. Reference: ISO 22196:2007(E). Plastics—Measurement of antibacterial activity on plastics surfaces.

2. Introduction:

A defined surface area is treated with the product and after 5 min contaminated with the tested microorganism.

3. The Product:

Product Name
Gloves treated with 5%
PurePurge

4. Acceptance Criteria:

The average U0 is: 6.2×10³≦U0≦2.5×10⁴.

The average Ut ≧6.2×10¹.

5. Test Organisms:

Esherichia coli ATCC 8739

Staphylococcus aureus ATCC 6538

6. Media:
TSB (Tryptic Soy Broth)
PBS (phosphate buffer saline)
TSA (Tryptic Soy Agar) BP (Baird Parker Agar) TBX Medium
Neutralizer:
LB (Modified Letheen Broth)

7. Preparation of the test organism:

An overnight incubated culture of each of the target organisms will be grown in TSB at 370 C for a minimum of 18 hours. The overnight culture will be adjusted to give a bacterial concentration of 2.5×10⁵ cfu/ml to 10×10⁵ cfu/ml using PBS. A serial dilution of the inoculum will be prepared and plated out on TSA to obtain an initial inoculum count. The plates will be incubated at 37° C. for 24 hours.
8. Test specimens: Flat, 50 mm×50 mm sheets of the Product.
9. Control specimens: Flat, 50 mm×50 mm Cutouts from a stomacher bag. Film: Cutouts from a stomacher bag (40 mm×40 mm)

10. Interfering substance:

Dirty Conditions—3.0 g/l bovine albumin

11. TEST OPERATION:
The tests were performed in triplicate.
9 test specimens were each placed into a separate sterile petri dish.
3 specimens were of the test specimens.
6 specimens were of the control specimens.

After the exposure period, each of the specimens was inoculated with 0.4 ml of the tested microorganism suspension. Each specimen was covered with a piece of film. The film was gently pressed down, so that the test inoculum would spread to the edges. Each petri dish was covered with the lid. 3 of the untreated and inoculated specimens were held for 5 minutes and then the bacteria were recovered. The other petri dishes (3 test and 3 control specimens) were incubated at 35° C. for 5 min.

12. Recovery: the specimens were transferred to individual containers, containing 10 ml of neutralizing broth. The containers were thoroughly shaken. The survival of the microorganisms after incubation was determined by using standard microbiological serial dilutions, pour plate count procedure and selective media plates (TBX for Esherichia coli). The plates were incubated at 37° C. for 24 hours. The number of microorganisms recovered after incubation was calculated. The results are shown below.

13. Study controls: The results are also shown below. Interfering substance sterility control: The interfering substance was cultured, incubated and visually examined for growth.

The acceptance criterion for this study control is lack of growth.

14. Carriers' sterility control:

Three uninoculated carriers were added to TSB medium, incubated and visually examined for growth. The acceptance criterion for this study control is lack of growth.

15. Results:

| | U0 | Ut | At | R |
|---|---|---|---|---|
| E. coli | 4.17 | 4.36 | 2.75 | 1.61 |
| ATCC 8739 | | | | |
| Staphylococcus | 3.34 | 3.42 | <1 | >3 |
| aureus | | | | |
| ATCC 6538 | | | | |

Abbreviations:
R—The antibacterial activity.(log reduction).
U0—The average of the common logarithm of the number of viable bacteria, in cells/cm², recovered from the untreated test specimens immediately after inoculation.
UT—The average of the common logarithm of the number of viable bacteria, in cells/cm2, recovered from the untreated test specimens after 5 min.
At—The average of the common logarithm of the number of viable bacteria, in cells/cm², recovered from the treated test specimens after 5 min.

| | | |
|---|---|---|
| Control | Control 1 | Control 1 |
| Interfering substance sterility | No Growth | No Growth |
| Control Specimens sterility | No Growth | No Growth |

16. Conclusion:

In reference to ISO 22196, the product Gloves treated with 5% PurePurge, batch No lab/281013/5, possess bactericidal activity on surfaces under dirty conditions at 5 min for referenced strains of Escherichia coli, Staphylococcus aureus.

Notably, the same conditions were used to test for growth after 24 hours. The following shows the results for 24 hours.

17. Results:

| | U0 | Ut | At | R |
|---|---|---|---|---|
| E. coli | 4.18 | 4.34 | <1 | >4 |
| ATCC 8739 | | | | |
| Staphylococcus | 4.01 | 4.48 | <1 | >4 |
| aureus | | | | |
| ATCC 6538 | | | | |

Abbreviations:
R—The antibacterial activity.(log reduction).
U0—The average of the common logarithm of the number of viable bacteria, in cells/cm², recovered from the untreated test specimens immediately after inoculation.
UT—The average of the common logarithm of the number of viable bacteria, in cells/cm², recovered from the untreated test specimens after 24 h.
At—The average of the common logarithm of the number of viable bacteria, in cells/cm², recovered from the treated test specimens after 24 h.

| | | |
|---|---|---|
| Control | Control 1 | Control 1 |
| Interfering substance sterility | No Growth | No Growth |
| Control Specimens sterility | No Growth | No Growth |

18. Conclusion:

In reference to ISO 22196, the product Gloves treated with 5% PurePurge, batch No lab/281013/5, possess bactericidal activity on surfaces under dirty conditions at 24 h for referenced strains of Escherichia coli, Staphylococcus aureus.

### EXAMPLE 9

In a still further embodiment, the photodynamic compositions of the present invention are manufactured by combining the following components, as shown in the following table. Quantities are in percentages by weight.

| Ingredient | Weight % range |
|---|---|
| Water | 97 < c > 99% |
| tert-Butyl Hydroperoxide or | 0.5 < c > 1.0% |
| hydrogen peroxide | |
| Alkyl (C12-16)benzyl | 0.1 < c > 0.5% |
| dimethylammonium chloride | |
| or bromide | |
| Polyhexamethylene Biguanide | 0.1 < c > 0.5% |
| Hydrochloride, chlorhexidine | |
| digluconate or dihydrochloride | |
| Fragrance | 0 < c > 0.15% |
| PurePurge | 0.1 < c > 2% |
| alkylpolyglucoside | 0 < c > 1.0% |

### REFERENCES

All references cited herein are hereby incorporated by reference herein for all purposes.

Collins T J, et al., Angew Chem Int Ed., 2006, 45, 3974-7.

Hamblin, M R, et al., Appl Env Microbiol., 2005, 71, 6918-25

Moir, A. et al., Cell Mol Life Sci., 2002, 59(3) 403-9.

Nyokong, T., Coordination Chemistry Reviews, 2007, 251, 1707-22

Wilson M., et al., J. Antimicrob. Chemotherap., 1997, 40, 873-6;

Wilson M., et al., J. Antimicrob. Chemotherap., 1994, 33, 619-624;

Wilson M., et al., J. Med. Microbiol., 1995, 42, 62-66.

1. An aqueous composition comprising zinc or aluminum phthalocyanine, a carboxylic acid C₆ to C₁₁ and/or hydroxy-acid C₂ to C₆ either as the free acid or as its alkali metal salt; dimethyl sulfoxide and optionally a biological dye selected from oxazine dyes, thiazine dyes and/or triarylmethyl containing dyes, wherein the composition treats or inhibits infectious disease caused by pathogenic microorganism in humans and animal.

2. The aqueous composition of claim 1, wherein the pathogenic microorganism is selected from bacteria, fungi, algae, yeast, bacterial or fungal spores.

3. The aqueous composition of claim 1, wherein the aluminum or zinc phthalocyanine is in an amount ranging from about 0.03% to about 0.5%, the sodium octanoate is in an amount ranging from about 0.01% to about 0.5%, the biological dye is in an amount ranging from about 0.1% to about 0.5%, the dimethyl sulfoxide in an amount from about 20% to about 30% and lactic acid in an amount from about 0.20% wt/vol to about 1.5% wt/vol.

4. The aqueous composition of claim 1, wherein the biological dye is selected from Benzo[a]phenoxazin-7-ium, 5-amino-9-(diethylamino)-, sulfate (Nile Blue); 3,7-Bis(dimethylamino)phenothiazin-5-ium chloride (methylene blue or methylthioninium chloride); 8-Dimethylamino-2,3-benzophenoxazine hemi(zinc chloride) salt (Meldola Blue); Phenoxazin-5-ium, 3-amino-7-(diethylamino)-2-methyl-, chlorozincate (Brilliant Cresyl Blue); Phenothiazin-5-ium, 3-amino-7-(dimethylamino)-2-methyl, chloride (1:1) (Toluidine Blue); [7-(dimethylamino)-4-nitrophenothiazin-3-ylidene]-dimethylazanium chloride (Methylene Green); Methanaminium, N-[4-[[4-(dimethylamino)phenyl]phenylmethylene]-2,5-cyclohexadien-1-ylidene]-N-methyl-, chloride (1:1) (Victoria Green,); Ethanaminium, N-[4-[[4-(diethylamino) phenyl]phenylmethylene]-2,5-cyclohexadien-l-ylidene]-N-ethyl-, sulfate (Brilliant Green); Methanaminium, N-[4-[[4-(dimethylamino) phenyl]phenylmethylene]-2,5-cyclohexadien-1-ylidene]-N-methyl-, chloride (Malachite Green); 4-[(4-Aminophenyl)-(4-imino-1-cyclohexa-2,5-dienylidene)methyl]aniline hydrochloride (Fuschine) and 4,4′-[(4-imino-2,5-cyclohexadien-1-ylidene)methylene]bisbenzenamine monohydrochloride (Basic Fuschine).

5. The aqueous composition of claim 1, further comprising hydrogen peroxide or tertiary-butylhydroperoxide and optionally at least one cationic microbiocide.

6. The aqueous composition of claim 5, wherein the cationic microbiocide is selected from guanidine salts or quaternary ammonium salts.

7. The aqueous composition of claim 6, wherein the guanidine salts is selected from chlorhexidine digluconate, dihydrochloride and diacetate; hexamethylenebis(ethylhexyl)biguanide dihydrochloride; oxocyclohexadienylideneaminoguanidine thiosemicarbazone; bis(chlorophenylamidino)piperazinedicarboxamidine dihydrochloride or polyhexamethylenebiguanidine hydrochloride.

8. The aqueous composition of claim 6, wherein the quaternary ammonium salts is a member selected from the group consisting of alkylbenzyldimethylammonium chloride, alkyl(C12-16)dimethylbenzyl ammonium chloride, benzylhexyldimethylammonium chloride, benzyloctyldimethylammonium chloride, benzyldecyldimethylammonium chloride, benzyldodecyldimethyl ammonium chloride, benzyltetradecyldimethylammonium chloride and benzyloctadecyldimethylammonium chloride.

9. A kit for cleansing a surface, the kit including the aqueous composition according to claim 1 and at least one applicator for applying the composition.

10. The kit of claim 9, wherein the applicator includes an absorbent material and the composition absorbed therein.

11. A method for combating micro-organisms in or on organic or inorganic substrates, the method comprising treating the substrates with a composition according to claim 1 in the presence of oxygen and water and while irradiating with visible and/or infrared light.

12. The method according to claim 11, wherein the surface is contaminated with one or more species of micro-organisms including bacteria, fungi, algae, yeasts, bacterial and fungal spores.

13. The method according to claim 11, further comprising hydrogen peroxide or tertiary-butylhydroperoxide and optionally at least one cationic microbiocide.

14. The method according to claim 13, wherein the surface is contaminated with one or more species of micro-organisms including bacteria, fungi, algae, yeasts, bacterial and fungal spores.

15. A method for disinfecting a surface, the method comprising applying a composition according to claim 1 and exposing such surface to electromagnetic radiation having a wavelength in the range from ultraviolet to infrared.

16. The method of claim 15, wherein the aqueous composition further comprises hydrogen peroxide, tertiary-butylhydroperoxide and/or at least one cationic microbiocide, wherein the at least one cationic microbiocide is a guanidine salt or a quaternary ammonium salt.

17. The method of claim 16, wherein the guanidine salts is selected from chlorhexidine digluconate, dihydrochloride and diacetate; hexamethylenebis(ethylhexyl)biguanide dihydrochloride; oxocyclohexadienylideneaminoguanidine thiosemicarbazone; bis(chlorophenylamidino)piperazinedicarboxamidine dihydrochloride or polyhexamethylenebiguanidine hydrochloride.

18. The method of claim 16, wherein the quaternary ammonium salts is a member selected from the group consisting of alkylbenzyldimethylammonium chloride, alkyl(C12-16)dimethylbenzylammonium chloride, benzylhexyldimethylammonium chloride, benzyloctyldimethylammonium chloride, benzyldecyldimethylammonium chloride, benzyldodecyldimethylammonium chloride, benzyltetradecyldimethylammonium chloride and benzyloctadecyldimethylammonium chloride.
